(19) United States
(12) Patent Application Publication
(10) Pub. No.: US 2025/0277175 A1
(43) Pub. Date: Sep. 4, 2025

(54) SINGLE-CHAMBER MICROBIAL ELECTROLYSIS CELL SYSTEM WITHOUT ION EXCHANGE MEMBRANE AND METHOD FOR PRODUCING CLEAN GAS SUCH AS HYDROGEN AND BIOGAS USING SAME

(71) Applicant: PUSAN NATIONAL UNIVERSITY INDUSTRY-UNIVERSITY COOPERATION FOUNDATION, Busan (KR)

(72) Inventors: Jung Rae KIM, Busan (KR); Min Soo KIM, Busan (KR)

(21) Appl. No.: 19/023,826

(22) Filed: Jan. 16, 2025

(30) Foreign Application Priority Data
Feb. 29, 2024 (KR) 10-2024-0030174
Nov. 26, 2024 (KR) 10-2024-0171430

(51) Int. Cl. C12M 1/107; C12M 1/06; C12M 1/12; C12M 1/34; C12M 1/42; C12M 3/00

(52) U.S. Cl. CPC C12M 21/04; C12M 23/06; C12M 23/42; C12M 27/02; C12M 35/02; C12M 41/12; C12M 41/32; C12M 41/34

(57) ABSTRACT

An embodiment of the disclosure provides a system capable of shortening the start-up time of hydrogen production of a microbial electrolysis cell through determining the step-by-step voltage application time, monitoring electrochemically active microbial colonies, and inhibiting technology of methane-converting bacteria for hydrogen production, and a method for producing hydrogen and biogas, in order to shorten the stabilization culture period of electroactive microorganisms through a single-chamber microbial electrolysis cell system.

### CROSS-REFERENCE TO RELATED APPLICATION

This application claims the priority of Korean Patent Application No. 10-2024-0030174, filed on Feb. 29, 2024, and Korean Patent Application No. 10-2024-0171430, filed on Nov. 26, 2024, in the Korean Intellectual Property Office, the disclosure of which is incorporated herein by reference.

### BACKGROUND

The disclosure relates to a single-chamber microbial electrolysis cell system without an ion exchange membrane and a method for producing clean gas including hydrogen and biogas using the same, and more specifically, to a single-chamber microbial electrolysis cell system utilizing a technology for determining the step-by-step voltage application time, monitoring electrochemically active microbial colonies, and inhibiting methane-converting bacteria for hydrogen production in order to shorten the stabilization culture period of a single-chamber microbial electrolysis cell, and a method for producing clean gas including hydrogen and biogas using the same. In addition, the disclosure provides a technology that may be applied to a post-treatment process of an anaerobic digestion process, thereby increasing the efficiency and applicability of biogas production, and simultaneously achieving wastewater treatment and energy recovery.

Microbial electrolysis cells have been actively studied recently because they are applicable to the treatment of high-concentration wastewater containing organic contaminants such as acetate and volatile fatty acids (VFAs), which are difficult to produce hydrogen with existing fermentation processes, and produce hydrogen.

In general, a two-chamber microbial electrolysis cell equipped with an ion exchange membrane between a cathode chamber and an anode chamber is widely applied, but a single-chamber microbial electrolysis cell without an ion exchange membrane is more applicable due to less contamination of the ion exchange membrane, electrical resistance, and cost-effective reactor configuration.

To rapidly produce hydrogen by operating a single-chamber microbial electrolysis cell without an ion exchange membrane, monitoring the organic matter decomposition performance of a specific electroactive microorganisms distributed in a reactor is essential. Simultaneously it is required to control the externally applied voltage and hydrogen production as well as to appropriately inhibit a specific microorganisms that consume the produced hydrogen to produce methane from the decomposition of organic matter.

However, most existing single-chamber microbial electrolysis cell operations require a longer period of start-up time, from 1 month to over 1 year, for hydrogen production and stabilization of specific electrochemically active microbial colonies. There is insufficient technology development related to shortening the hydrogen production start-up time of single-chamber microbial electrolysis cells.

Many previous studies have the disadvantage of requiring a long period of start-up time for the entire process (pre-process) for hydrogen production, such as cultivating specific electrochemically active microbial colonies that decompose organic matter for a long period of time in microbial fuel cells (MFCs) and then release electrons, or applying low voltage (<0.6 V, based on total cell voltage) for a long period of time to induce stabilization of microbial electrolysis cells.

### SUMMARY

An aspect of the disclosure is providing a technology that may shorten the start-up time required for hydrogen production through single-chamber microbial electrolysis cell operation and the stabilizing specific electrochemically active microorganisms.

The disclosure is to provide a carbon-neutral and environmentally friendly technology that may realize the production of hydrogen and biogas containing a large amount of hydrogen while treating high-concentration organic wastewater.

The disclosure is to solve the previous disadvantages, such as ohmic loss due to use of an ion exchange membrane, difficulty in scale-up due to the complexity of a reactor structure, biofouling of a separation membrane, and increase in the reactor price due to a high-cost separation membrane by not using an ion exchange membrane.

The aspect of the disclosure is not limited to that mentioned above, and other aspects not mentioned will be clearly understood by those skilled in the description below.

An embodiment of the disclosure provides a single-chamber microbial electrolysis cell system without an ion exchange membrane, the single-chamber microbial electrolysis cell system including: a power supply unit; a microbial electrolysis chamber connected to the power supply unit; an electrode unit located within the chamber; and a water bath connected to the chamber and used for maintaining a temperature, wherein the chamber includes: a gas collection unit for confirming biogas production within the chamber; an organic matter supply unit for supplying organic matter into the chamber; a gas supply unit for supplying an inert gas into the chamber; a sampling unit for confirming consumption of organic matter within the chamber; a solution replacement unit for replacing organic matter within the chamber; and a stirring unit for stirring within the chamber, wherein the electrode unit includes: an anode electrode (anode of organic contaminant) and a cathode electrode (cathode of proton to hydrogen) installed opposite each other within the chamber; and a reference electrode located next to the anode or cathode electrodes.

In an embodiment of the disclosure, the chamber may be cylindrical or cassette-shaped. If the chamber is cassette-shaped, the cassette-shaped chamber may expose only the cathode electrode to the air to specifically inhibit methanogens formed on the electrode or allow the anode electrode and the cathode electrode to be separately removable or replaceable for regular electrode maintenance.

In an embodiment of the disclosure, to monitor biogas generated inside the chamber through the gas collection unit and concomitantly inhibit methanogens formed on the cathode electrode, a method of exposing the cathode electrode to the air, a method of replacing an organic matter medium to remove suspended methanogen in the case of batch operation, a method of removing a solution-phase methanogen by operating HRT within 24 hours in the case of continuous operation, or a method of supplying a chemical inhibitor or antibiotic into the chamber may be performed.

In an embodiment of the disclosure, a gas-liquid separator may be combined on one side of the chamber.

In an embodiment of the disclosure, the gas-liquid separator may reduce the residence time of hydrogen produced within the chamber, thereby reducing the activity of microorganisms that convert the produced hydrogen to methane.

Another embodiment of the disclosure provides a method for producing hydrogen and biogas using a single-chamber microbial electrolysis cell system without an ion exchange membrane, the method including: supplying organic matter to a chamber through an organic matter medium without applying an external voltage; forming an electrochemically active microbial group by applying an external voltage to the chamber; inhibiting methanogens formed on a cathode electrode within the chamber; and producing hydrogen and biogas.

In an embodiment of the disclosure, in supplying organic matter to a chamber through an organic matter medium without applying an external voltage, anaerobic digestion conditions may be formed inside the chamber so that hydrogen and biogas start to form in the reactor. For the initial anaerobic condition of the chamber, an inert gas was purged into the liquid phase of the chamber at a rate of 50 ml/min or less for 15 to 20 minutes.

In an embodiment of the disclosure, the organic matter was suppled to a chamber through a medium without applying an external voltage. The temperature of the chamber may be maintained at 20 to 35° C. in this stage.

In an embodiment of the disclosure, the external voltage may be applied at 0.6 to 1.2 V using a two-electrode voltage application scheme.

In an embodiment of the disclosure, the inhibiting of the methanogens formed on the cathode electrode within the chamber may be performed through a method of replacing a medium containing organic matter, a method of removing a solution-phase methanogen by operating HRT within 24 hours in the case of continuous operation, a method of exposing a hydrogen-producing cathode electrode to the air for 10 minutes to 1 hour, or a method of supplying a chemical inhibitor or antibiotic.

In an embodiment of the disclosure, the chemical inhibitor may be implemented from the group including sodium 2-bromoethanesulfonate, 2-bromoethanesulfonate, Iodopropane, and lumazine, and the antibiotic may be selected from the group including neomycin sulfate, 2-chloroethane sulfonate, and 8-aza-hypoxanthine.

In an embodiment of the disclosure, in the hydrogen and biogas production, a method of monitoring the potential of an anode electrode based on a reference electrode in the chamber may be applied to check the concentration of organic matter and followed by replacing the medium to provide organic matter.

In an embodiment of the disclosure, in the hydrogen and biogas production, controlling the content of carbon dioxide, methane, and hydrogen of the biogas produced from the single-chamber microbial electrolysis cell system without an ion exchange membrane, by control of applied potential, temperature of the chamber, HRT (residence time of reactants in the chamber), or the application of a method of inhibiting the activity of methanogen and homoacetogen to produce suitable biogas for the intended purpose of applications.

In an embodiment of the disclosure, in the supplying of the organic matter to a chamber by an organic matter medium without applying an external voltage, if the organic matter is a liquid containing an organic acid and a gas containing carbon dioxide and methane, organic contaminants contained in the liquid containing an organic acid may be removed within the chamber, and at the same time, the carbon dioxide of the gas is combined with hydrogen produced in the single-chamber microbial electrolysis cell system and converted into methane; and highly soluble impurities including sulfur compounds, silicic acid, and odorous substances further contained in the gas may be removed while passing through the liquid within the chamber, so that the content ratio of hydrogen and methane of the biogas manufactured in the single-chamber microbial electrolysis cell system increases, thereby improving the quality.

According to an embodiment of the disclosure, the time required for hydrogen production and stabilization of specific electrochemically active microbial culture through single-chamber microbial electrolysis cell operation can be shortened.

It is possible to provide a carbon-neutral and environmentally friendly technology that can realize the production of hydrogen and biogas with high hydrogen content while treating high-concentration organic wastewater such as wastewater and food waste sludge.

It is possible to overcome thermodynamic limitations, produce hydrogen from various organic matter at room temperature and pressure, consume low electric energy (0.6-1.2V) compared to a water electrolysis process (>1.23V theoretical value), and furthermore since an ion exchange membrane is not used, it is possible to solve the disadvantages such as Ohmic loss due to a separation membrane, difficulty in scale-up due to the complexity of a reactor structure, biofouling of a separation membrane, and increase in the reactor price due to a high-cost separation membrane.

Currently, high-concentration organic wastewater such as sewage water and food waste sludge is converted into biogas such as methane through anaerobic digestion. However, since the methane content of the biogas from the anaerobic digestion process is only 40-60% at most, it has the disadvantage of low calorific value for direct replacement of city gas or use in combined heat and power generation. The technology proposed in the disclosure can convert organic acids, which are difficult to additionally convert into methane in the anaerobic digestion process itself, into hydrogen, so the same can be used for improving the gas composition of biogas, improving the calorific value during combustion, and as a pretreatment process for the subsequent hydrogen separation process. In addition, biogas from the anaerobic digestion process containing sulfur compounds, siloxane, and odorous substances can be passed through the liquid phase of a microbial electrolytic cell to remove highly soluble contaminants, thereby contributing to improving the quality of biogas.

The effects of the disclosure are not limited to the effects described above, and should be understood to include all effects that are inferable from the configuration of the disclosure described in the detailed description or claims of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1A is a schematic view of a single-chamber microbial electrolysis cell system without an ion exchange membrane according to an embodiment of the disclosure, and FIG. 1B is an actual appearance of a lab-scale single-chamber electrolysis cell system;

FIG. 2A is a schematic view of a cylindrical single-chamber microbial electrolysis cell system without an ion exchange membrane according to an embodiment of the disclosure, and FIG. 2B is an actual view of a bench-scale single-chamber electrolysis cell system;

FIG. 3A is a schematic view of a cassette-shaped single-chamber microbial electrolysis cell system without an ion exchange membrane, in which an anode electrode and a cathode electrode are separably attachable and detachable as needed according to an embodiment of the disclosure, and FIG. 3B is a schematic view of a bench-scale single-chamber electrolysis cell system;

FIG. 4 is a schematic view of a method for producing clean gas including hydrogen and biogas using a single-chamber microbial electrolysis cell system without an ion exchange membrane according to an embodiment of the disclosure;

FIGS. 5A, 5B, and 5C show the results of (5A) microbial colonies attached to an anode electrode at a phylum level, (5B) microbial colonies attached to a cathode electrode, and (5C) microbial colonies attached to an anode electrode and cathode electrode at a genus level after operation of a single-chamber type microbial electrolysis cell system without an ion exchange membrane according to an embodiment of the disclosure (based on NGS analysis);

FIGS. 6A and 6B are results of hydrogen production in biogas production in a single-chamber type microbial electrolysis cell system without an ion exchange membrane according to an embodiment of the disclosure (6A) without application and (6N) after application of the technology proposed in the disclosure;

FIG. 7 is a schematic view of a single-chamber microbial electrolysis cell system without an ion exchange membrane, coupled with a gas-liquid separator, according to an embodiment of the disclosure; and

FIG. 8 is a schematic view of an anaerobic digestion tank connected to a single-chamber microbial electrolysis cell system according to an embodiment of the disclosure, where anaerobic digestion conditions are formed.

### DETAILED DESCRIPTION

Hereinafter, the disclosure will be described with reference to the accompanying drawings. However, the disclosure may be implemented in various different forms, and therefore is not limited to the embodiments described herein. In addition, in order to clearly describe the disclosure in the drawings, parts that are not related to the description are omitted, and similar parts are given similar drawing reference numerals throughout the specification.

In the entire specification, when a part is said to be “connected (linked, contacted, coupled)” to another part, this includes not only the case where it is “directly connected” but also the case where it is “indirectly connected” with another member in between. In addition, when a part is said to “include” a certain component, this does not mean that other components are excluded unless otherwise specifically stated, but that other components may be additionally provided.

The terms used in this specification are used only to describe specific embodiments and are not intended to limit the disclosure. The singular expression includes the plural expression unless the context clearly indicates otherwise. In this specification, the terms “include” or “have” are intended to specify the presence of a feature, number, step, operation, component, part, or combination thereof described in the specification, but should be understood as not excluding in advance the possibility of the presence or addition of one or more other features, numbers, steps, operations, components, parts, or combinations thereof.

Hereinafter, Embodiments of the Disclosure Will be Described in Detail with Reference to the Accompanying Drawings.

A single-chamber microbial electrolysis cell system without an ion exchange membrane according to an embodiment of the disclosure will be described.

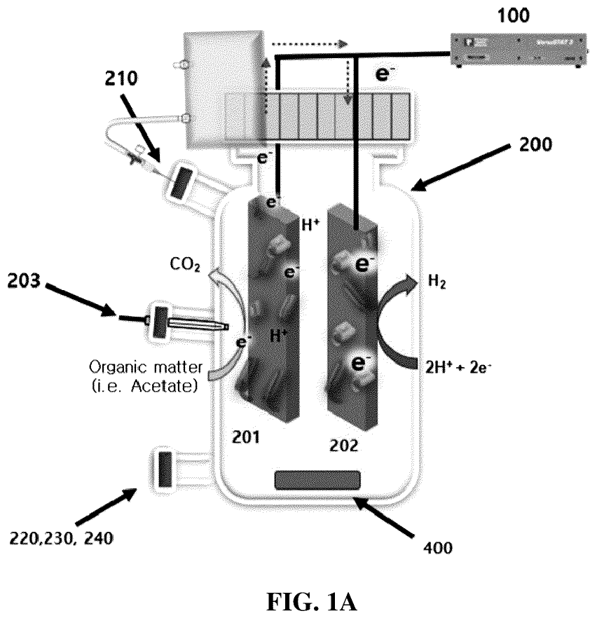

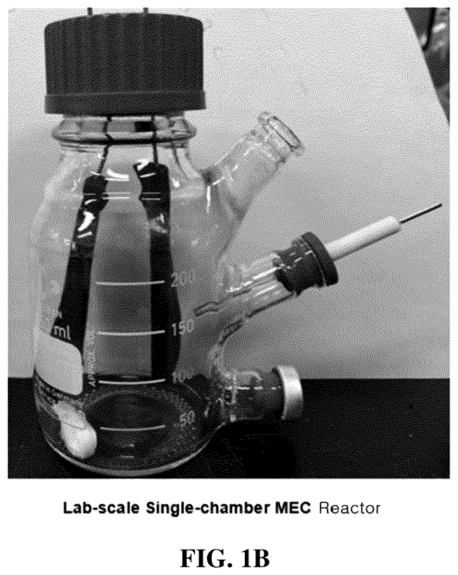

FIG. 1A is a schematic view of a single-chamber microbial electrolysis cell system without an ion exchange membrane according to an embodiment of the disclosure, and FIG. 1B is an actual appearance of a lab-scale single-chamber electrolysis cell system.

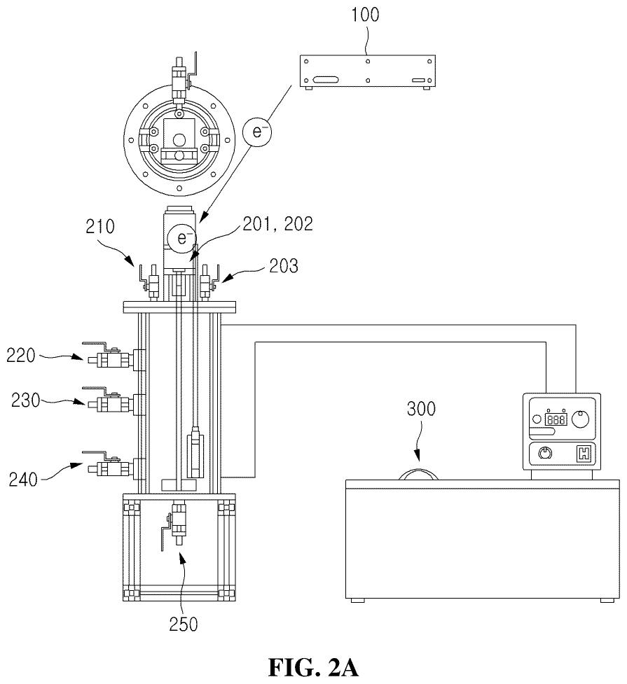

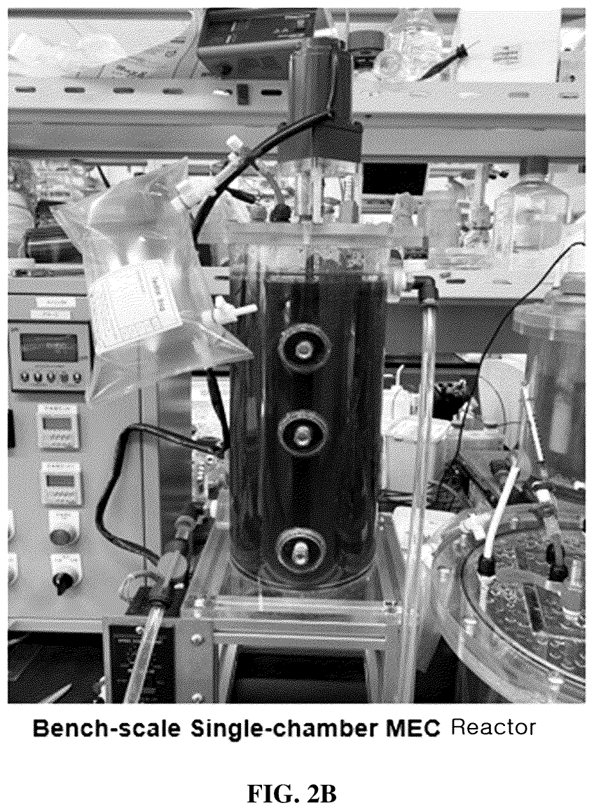

FIG. 2A is a schematic view of a cylindrical single-chamber microbial electrolysis cell system without an ion exchange membrane according to an embodiment of the disclosure, and FIG. 2B is an actual view of a bench-scale single-chamber electrolysis cell system.

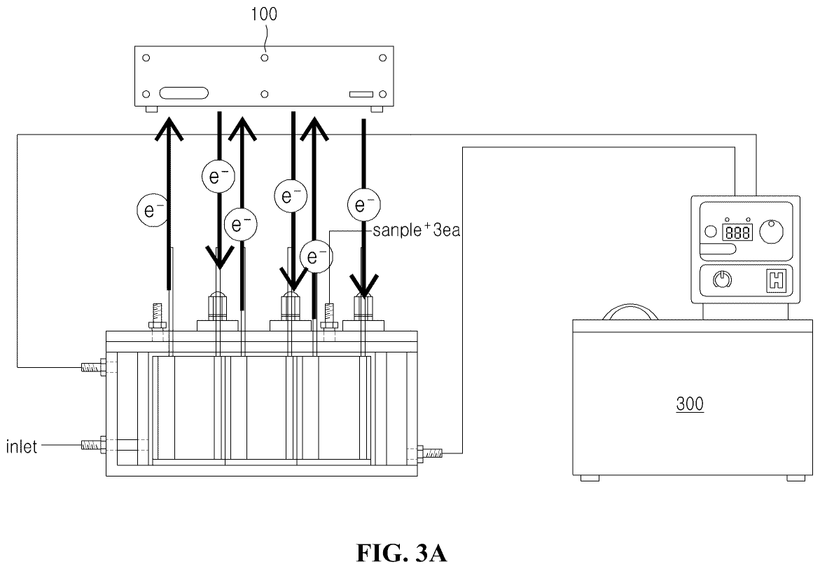

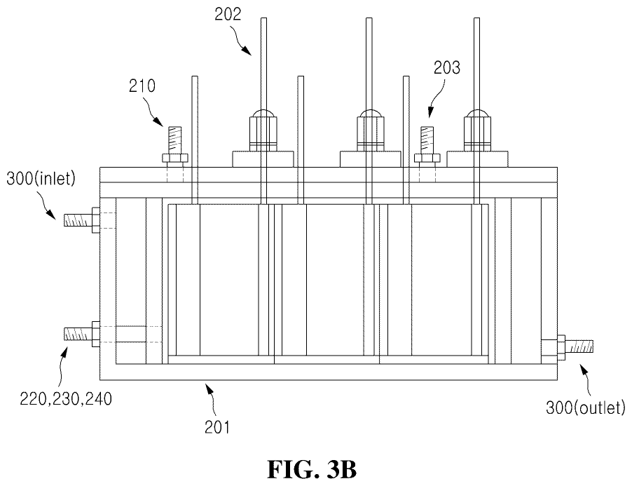

FIG. 3A is a schematic view of a cassette-shaped single-chamber microbial electrolysis cell system without an ion exchange membrane according to an embodiment of the disclosure, and FIG. 3B is a schematic view of a bench-scale single-chamber electrolysis cell system.

Referring to FIGS. 1A and 1B to FIGS. 3A and 3B, a single-chamber microbial electrolysis cell system without an ion exchange membrane according to an embodiment of the disclosure may include: a power supply unit 100; a microbial electrolysis chamber 200 connected to the power supply unit 100; an electrode unit located within the chamber 200; and a water bath 300 connected to the chamber 200 and used to maintain a temperature, wherein the chamber 200 includes: a gas collection unit 210 for confirming biogas production within the chamber 200; an organic matter supply unit 220 for supplying organic matter into the chamber 200; a gas supply unit 230 for supplying an inert gas into the chamber 200; a sampling unit 240 for confirming consumption of organic matter within the chamber 200; a solution replacement unit 250 for replacing organic matter within the chamber 200; and a stirring unit 400 for stirring inside the chamber, and the electrode unit may include: an anode electrode 201 and a cathode electrode 202 installed opposite each other within the chamber 200; and a reference electrode 203 located next to the anode electrode 201 or the cathode electrode 202.

The chamber may be cylindrical or cassette-shaped, and if the chamber is cassette-shaped, the cassette-shaped chamber may expose only the cathode electrode to the air to inhibit methanogens formed on the cathode electrode, or allow the anode electrode and the cathode electrode to be separably attachable and detachable for electrode maintenance.

Meanwhile, in order to monitor biogas generated inside the chamber through the gas collection unit and inhibit methanogens formed on the cathode electrode, a method of exposing the cathode electrode to the air, a method of replacing an organic matter medium in the case of batch operation, a method of removing a solution-phase methanogen by operating HRT within 24 hours in the case of continuous operation, or a method of supplying a chemical inhibitor or antibiotic into the chamber may be performed.

In order to monitor the biogas produced at this time, gas chromatography (GC) equipment may be connected to the gas collection unit in the reactor to check the real-time gas composition, and the amount of gas produced may be checked in real-time by connecting to a respirometer.

At this time, the amount of methanogen formed on the cathode electrode may be inferred by checking the amount of biogas produced using the method described above, and the methods described above may be performed to inhibit the methanogen formed at this time.

Accordingly, the gas collection unit of the single-chamber microbial electrolysis cell system without an ion exchange membrane of the disclosure is further connected to a monitoring unit including a gas chromatography device, so that a user can check the amount of biogas produced through the monitoring unit and infer the amount of methanogen through this, and then the user can immediately proceed with a method of exposing the cathode electrode to the air to inhibit the formed methanogen.

Alternatively, the user may use a method of replacing an organic matter medium when operating the reactor in batch mode, or a method of removing solution-phase methanogen colonies by operating HRT within 24 hours when operating the reactor in continuous mode.

Alternatively, the user may perform a method of supplying chemical inhibitors or antibiotics into the chamber.

At this time, it does not matter whether one or more of the above-mentioned methods are performed.

Meanwhile, microbial electrolysis cells have been actively studied recently because they may be applied to the treatment of high-concentration wastewater containing organic acids, such as acetate, which are difficult to produce hydrogen with existing fermentation processes, and may produce hydrogen and biogas containing a large amount of hydrogen.

At this time, the microbial electrolysis cell is largely divided into a two-chamber reaction system and a single-chamber reaction system. In the case of the two-chamber reaction system, the reaction system is divided into an anode chamber and a cathode chamber by an ion exchange membrane, wherein the organic matter supplied by the electroactive microorganisms to the anode chamber is decomposed to generate electrons and hydrogen ions (H⁺), electrons and hydrogen ions (H⁺) thus generated move to the cathode electrode through an external circuit, the hydrogen ions (H⁺) move to the cathode chamber through the ion exchange membrane, and a hydrogen generation reaction occurs at the cathode electrode.

Although a two-chamber microbial electrolysis cell equipped with an ion exchange membrane between the cathode chamber and the anode chamber is widely applied, the disclosure pertains to a single-chamber microbial electrolysis cell without an ion exchange membrane due to contamination and electrical resistance of an ion exchange membrane, complexity of a reactor structure, and the increased reactor price.

Specifically, the disclosure relates to a technique for determining the stepwise voltage application time, monitoring an activity of electrochemically active microorganisms, and inhibiting the methane conversion bacteria for hydrogen production in order to shorten the stabilization culture period of a single-chamber microbial electrolytic cell.

In the case of the two-chamber type, microorganisms are inoculated and cultured only in an anode chamber. The advantage of this case is that the chambers can be distinguished, so the microorganisms can be grown only in the anode chamber. However, the disadvantage is that the performance of a membrane or ion exchange membrane deteriorates over time. In addition, the reactor structure becomes complicated due to the use of the membrane or ion exchange membrane, making it difficult to change, and the production cost increases.

Therefore, disclosed herein is a reactor by introducing microorganisms into a single chamber without an ion exchange membrane.

It is important to selectively culture specific electrochemically active microbial colonies on both an anode electrode and a cathode electrode.

In addition, since microorganisms cannot be formed only on the anode electrode, measures should be taken to selectively culture electrochemically active microorganisms on the anode electrode and cathode electrode, and among microorganisms cultured on the cathode electrode, methanogen is inhibited by the method presented above so that a lot of hydrogen can be produced.

Meanwhile, a single-chamber microbial electrolysis cell system without an ion exchange membrane may produce biogas containing CH₄ and CO₂ in addition to hydrogen production because there is no ion exchange membrane that separates the anode and cathode reactions of organic matter in the reactor.

In this process, specific microbial colonies that consume hydrogen, such as Methanogen (CO₂+4H₂-->CH₄+2H₂O) and Homoacetogen (2CO₂+4H₂-->CH₃COOH+2H₂O), may be involved, so if the two colonies are properly controlled, more stable biogas production containing hydrogen may be possible.

The content of carbon dioxide, methane, and hydrogen, which are the main components of biogas, may be controlled by the technology proposed in the disclosure to produce biogas with a high hydrogen content that can be used for combustion or gas power generation, or can be utilized as a pretreatment process to reduce the burden of the pressure sweep adsorption (PSA) process for producing high-purity hydrogen.

To this end, a gas-liquid separator 500 may be further coupled with one surface of the single-chamber of the single-chamber microbial electrolysis cell system without an ion exchange membrane according to the disclosure.

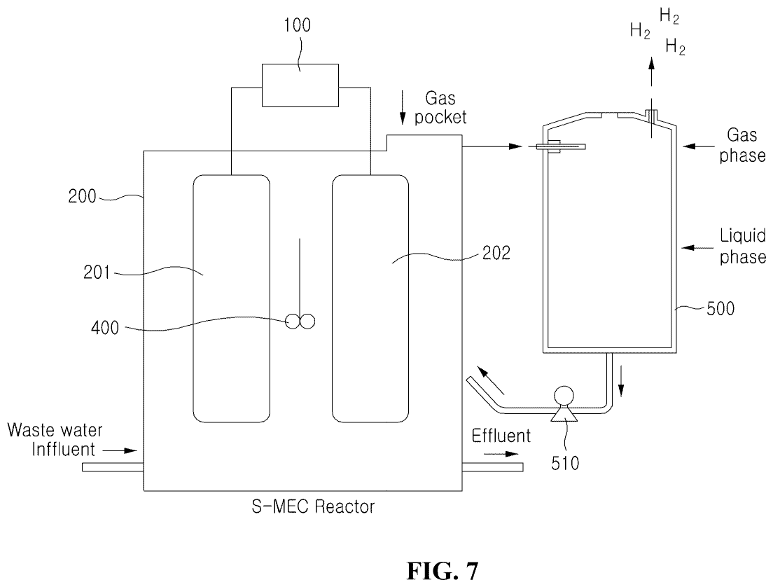

FIG. 7 is a schematic view of a single-chamber microbial electrolysis cell system without an ion exchange membrane, further coupled with a gas-liquid separator 500, according to an embodiment of the disclosure.

Referring to FIG. 7, it can be confirmed that a gas-liquid separator 500 is coupled with one surface of the chamber of the single-chamber microbial electrolysis cell system without an ion exchange membrane according to an embodiment of the disclosure.

As explained above, specific microbial colonies that consume hydrogen, such as Methanogen (CO₂+4H₂-->CH₄+2H₂O) and Homoacetogen (2CO₂+4H₂-->CH₃COOH+2H₂O), may interfere with stable hydrogen production in the reactor.

Accordingly, various methods to suppress Methanogen and Homoacetogen have been studied, and in particular, papers on a method of suppressing related enzymes by injecting chemicals such as 2-bromoethanesulfonate and chloroform have been reported; however, not only does it affect specific enzymes, but it also affects the activity of all microorganisms, which may reduce the performance of the entire reactor, and in particular, it is difficult to apply to large-scale reactors due to cost and environmental issues.

By physically suppressing Methanogen and Homoacetogen through the reactor structure and operation, the residence time of hydrogen produced through the microbial electrolysis cell reaction in the reactor can be minimized, and the method of suppressing the reaction in which Methanogen and Homoacetogen consume hydrogen can be utilized as much as possible, and the gas-liquid separator 500 can be used for this.

Specifically, the gas headspace of the microbial electrolysis cell is minimized (i.e., the liquid phase is filled to the top of the reactor), and the gas (biogas containing hydrogen) generated from the top of the reactor and the liquid flowing out are transferred to a gas-liquid separator (Gas/Liquid separator) to be separated into gas and liquid, and the gas can be captured at the top and the liquid can be collected at the bottom and returned to the microbial electrolysis cell. In this way, effective separation of the electrolyte and gas is possible, and in particular, it is possible to quickly separate the generated hydrogen from the microbial electrolysis cell chamber (reactor) to suppress methanogen or acetic acid producing bacteria.

At this time, if the generated hydrogen is not separated quickly, as mentioned above, hydrogen generated by methanogen or homoacetogen is recycled, which causes a problem of a decrease in the production speed and yield of hydrogen.

The design considerations for combining the gas-liquid separator 500 with the single-chamber microbial electrolysis cell system without an ion exchange membrane of the disclosure are as follows.

First, a gas pocket where the gas generated in a microbial electrolysis cell reactor is collected must be located at the top of an MEC reactor, and a gas pump must be connected so that a gas passed to the gas-liquid separator 500 can be collected in a gas storage at the top of the reactor.

In addition, a liquid pump 510 may be installed at the bottom of the gas-liquid separator 500 to recirculate an electrolyte (microbial medium) to minimize the loss of the electrolyte and maintain the continuity of the process.

Through this configuration, the gas produced is captured at the top of the gas-liquid separator 500, the captured gas is stored in the gas storage, and the liquid is introduced into the reactor from the bottom of the gas-liquid separator 500 by a liquid pump so that the reaction can continue.

In addition, a monitoring system is further connected to the outside of the reactor to track the potential of an anode electrode that changes according to the concentration of the substrate being injected, and at the same time, the pH range, temperature, and partial pressure maintained in the reactor may be measured.

Therefore, in the single-chamber microbial electrolysis cell system without an ion exchange membrane combined with the gas-liquid separator 500 of the disclosure, a certain space may be formed so that a gas pocket is formed at the top of a chamber.

In addition, a gas storage may be located at the top of the gas-liquid separator 500.

In addition, a liquid pump 510 is located at the bottom of the gas-liquid separator 500, and the liquid pump 510 may connect the gas-liquid separator 500 and the chamber.

Through the above-described configuration, the gas-liquid separator 500 of the disclosure may reduce the residence time of hydrogen produced in the chamber, thereby reducing the activity of microorganisms that consume the hydrogen produced in the chamber.

Hereinafter, a specific method of producing hydrogen using a single-chamber microbial electrolysis cell system without an ion exchange membrane having the above-described characteristics will be described.

### A Method for Producing Hydrogen and Biogas Using a Single-Chamber Microbial Electrolysis Cell System without an Ion Exchange Membrane According to an Embodiment of the Disclosure Will be Described

A method for producing clean gas, including hydrogen and biogas using a single-chamber microbial electrolysis cell system without an ion exchange membrane according to an embodiment of the disclosure may apply all of the contents described for the single-chamber microbial electrolysis cell system without an ion exchange membrane described above, and detailed descriptions of overlapping parts have been omitted, but even if the descriptions are omitted, they may be applied equally.

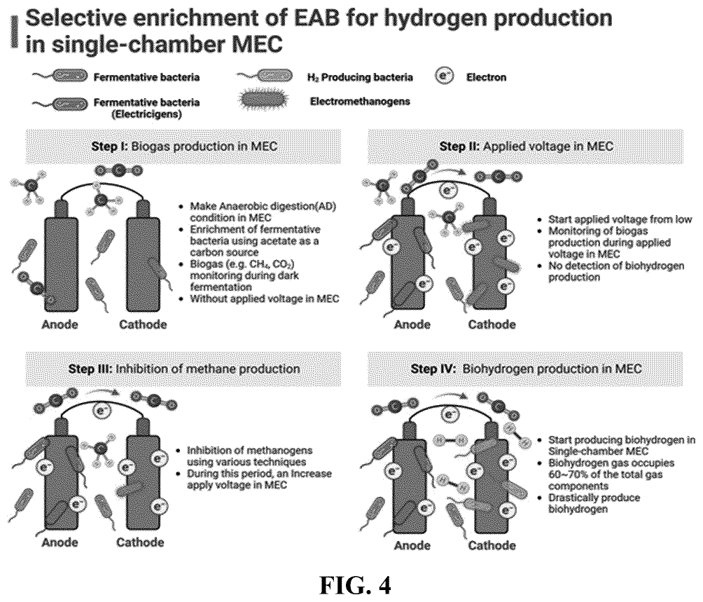

FIG. 4 is a schematic view of a method for producing clean gas including hydrogen and biogas using a single-chamber microbial electrolysis cell system without an ion exchange membrane according to an embodiment of the disclosure.

Referring to FIG. 4, a method for producing hydrogen and biogas using a single-chamber microbial electrolysis cell system without an ion exchange membrane according to an embodiment of the disclosure may include: supplying organic matter to a chamber through an organic matter medium without applying an external voltage; forming an electrochemically active microorganisms on the anode electrode by applying an external voltage to the chamber; inhibiting methanogens formed on a cathode electrode within the chamber; and producing hydrogen.

The First is Supplying Organic Matter to a Chamber Through an Organic Matter Medium without Applying an External Voltage.

In the supplying of the organic matter to the chamber through an organic matter medium without applying an external voltage, dark fermentation proceeds, and as this dark fermentation proceeds, organic matter is decomposed by microorganisms and used. Biogas is produced through this dark fermentation of a carbon source. Biogas may be formed by forming anaerobic digestion conditions within the chamber. The biogas produced at this time is mainly composed of methane and CO₂, and may contain impurities such as hydrogen sulfide (H₂S), moisture (H₂O), ammonia (NH₃), nitrogen (N₂), and oxygen (O₂).

The above anaerobic digestion conditions may be formed by supplying an inert gas into the chamber. Nitrogen or argon gas may be used as the inert gas, but this is not limited thereto.

The supply of the inert gas may be supplying at a rate of 50 ml/min or less for 15 to 20 minutes.

The temperature of the chamber may be maintained at 20 to 35° C.

Chemical substances included in the organic matter may include sodium acetate, NH₄Cl, NaCl, MgSO₄·7H₂O, KCl, or yeast extract, which are used as carbon sources, and may be used as a medium for microbial growth.

Secondary sedimentation sludge, wastewater from wastewater treatment plants, and mixed wastewater containing domestic wastewater and sewage may be used as mixed strain inoculation sources.

In order to monitor the biogas produced at this time, gas chromatography GC equipment and a gas collection unit in a reactor are connected to check the real-time gas composition. The amount of gas produced may be checked in real-time by connecting to a respirometer.

### The Next is Forming an Electrochemically Active Microorganisms on the Anode by Applying an External Voltage to the Chamber.

The external voltage may be a low voltage of 0.6 to 1.2 V applied using a two-electrode voltage application method. At the same time, the biogas produced may be monitored. Under the external voltage application conditions, the growth and formation of a specific electrochemically active microbial group that decomposes organic matter proceeds.

This may be a process of confirming the growth of microbial colonies that may grow under electron supply conditions while simultaneously applying a potential while forming a specific electrochemically active microbial group that releases electrons using organic matter.

At this time, the electrochemically active microbial colonies that grow and are formed may be formed on an anode electrode and cathode electrode surfaces, and may also be formed inside the medium.

The Next is Inhibiting Methanogens Formed on a Cathode Electrode within the Chamber.

In order to produce hydrogen on the cathode electrode, methanogens that produce methane gas formed on the cathode electrode must be inhibited. This is because, instead of producing hydrogen by reducing protons and electrons, a competitive reaction occurs where methanogen formed on the cathode electrode reduces carbon dioxide contained in biogas to electrons and hydrogen ions to produce methane gas.

The inhibiting of the methanogens located on a cathode electrode in the chamber may be performed through a method of exposing the cathode electrode to the air, a method of replacing an organic matter medium, a method of removing a solution-phase methanogen by operating HRT within 24 hours in the case of continuous operation, a method of exposing a hydrogen-producing cathode electrode to the air for 10 minutes to 1 hour, or a method of supplying a chemical inhibitor or antibiotic.

The method of replacing the organic matter medium is to prevent methanogens from growing in the reactor by replacing the medium quickly because the growth rate of methanogen is slow, wherein it is desirable to make replacement as quickly as possible.

The above chemical inhibitor or antibiotic may be supplied into the chamber, wherein the chemical inhibitor may be at least one inhibitor selected from the group including Sodium 2-bromoethanesulfonate, 2-bromoethanesulfonate, Iodopropane, and lumazine, and the antibiotic may be at least one antibiotic selected from the group including Neomycin sulfate, 2-chloroethane sulfonate, and 8-aza-hypoxanthine.

An operation scheme of this system may be divided into a continuous scheme and a batch scheme, and the medium can be changed through the batch scheme to enable rapid hydrogen production.

### The Next is Producing Hydrogen and Biogas.

The producing of the hydrogen and biogas may be performed by checking the concentration of organic matter while monitoring the potential of the anode electrode in the chamber based on a reference electrode in the chamber and replacing the organic matter medium.

When the organic matter is depleted, the potential of the anode electrode also decreases, so the experiment can be conducted while checking the tendency, and a method of monitoring the potential of the electrode may be to check the potential of the anode electrode that changes in real time using a voltameter or a Lab-view program. The potential of the anode electrode may be conducted while recognizing that it varies depending on the conductivity of the medium used, the material of the anode electrode and the material of the cathode electrode.

Through biogas monitoring, the start-up time of hydrogen production may be shortened by adding a methanogen inhibitor.

Meanwhile, by comprehensively considering the conditions mentioned above, in the producing of the hydrogen and biogas, the carbon dioxide, methane, and hydrogen contents of the produced biogas may be adjusted according to the voltage applied to the single-chamber microbial electrolysis cell system without an ion exchange membrane, the temperature of the chamber, HRT (the time the reactants remain in the chamber), or whether or not a method of inhibiting the activity of methanogen and homoacetogen is applied, so that biogas suitable for the intended use can be produced.

Meanwhile, in the supplying of the organic matter to the chamber through an organic matter medium without applying an external voltage, when the organic matter is a liquid containing an organic acid and a gas containing carbon dioxide and methane, organic contaminants contained in the liquid containing an organic acid are removed within the chamber, and at the same time, carbon dioxide of the gas is combined with hydrogen produced in the single-chamber microbial electrolysis cell system and converted into methane, and highly soluble impurities including sulfur compounds, silicic acid, and malodorous substances contained in the gas are removed while passing through the liquid within the chamber, so that the content ratio of hydrogen and methane of the biogas manufactured in the single-chamber microbial electrolysis cell system increases, thereby improving the quality.

At this time, improving the quality of the biogas means that the content of methane and hydrogen contained in the biogas is improved.

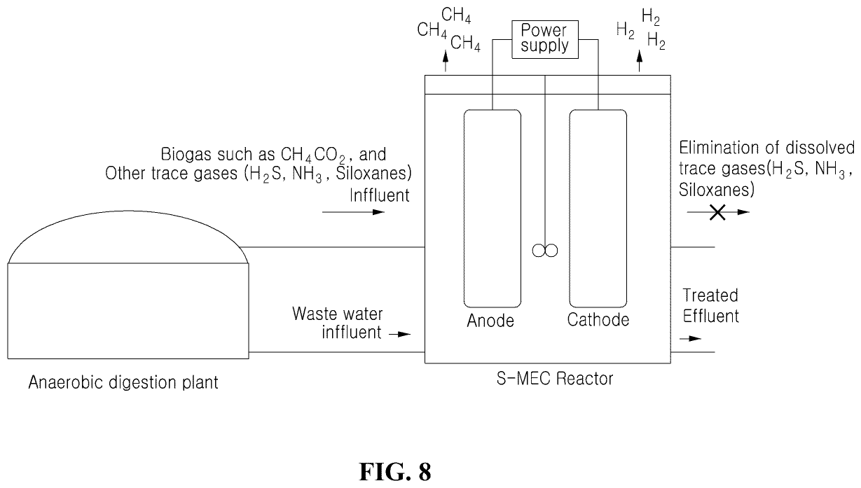

FIG. 8 is a schematic view of an anaerobic digestion tank connected to a single-chamber microbial electrolysis cell system according to an embodiment of the disclosure, where anaerobic digestion conditions are formed.

Referring to FIG. 8, an anaerobic digestion tank is connected to the front end, wherein at this time, the liquid phase containing organic acid of the anaerobic digestion tank and the exhaust gas containing carbon dioxide and methane are introduced into a single-chamber microbial electrolysis cell system without an ion exchange membrane to purify the liquid organic pollutants, and at the same time, the gaseous CO₂ is combined with the hydrogen produced in the microbial electrolysis cell to convert it into methane, and the highly soluble impurities including sulfur compounds, siloxanes, and odorous substances present in the exhaust gas of the anaerobic digestion tank are removed by passing them through the liquid phase, thereby improving the quality of the biogas.

Hereinafter, an experimental example of the disclosure will be described in detail.

### Experimental Example

TABLE 1

| | | Stabilization | | | | |
|---|---|---|---|---|---|---|
| | | method of | | | Hydrogen | |
| | | microbial | Start-up | Applied | production | |
| Reactor type | [symbol] | community | time | voltage (V) | rate | Ref |
| Ten Single- | The primary | Repeated | 25 day | 2.0 | 0.258 | [symbol] |
| chamber | sedimentation | fed-batch | | | [symbol] /day | [symbol] |
| bottle-shaped | tank from a | cycles | | | | [symbol] |
| | wastewater | under an | | | | [symbol] |
| | treatment | applied | | | | [symbol] |
| | plant | voltage of | | | | [symbol] |
| | | 0.8 V | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| Single- | Two-chamber | Operated | 29 day | 0.9 | 3.66 | [symbol] |
| chamber | Microbial | two-chamber | | | [symbol] /day | [symbol] |
| | fuel cell | MFC | | | | [symbol] |
| | (MFC) | | | | | [symbol] |
| | operated for a | | | | | [symbol] |
| | long period | | | | | [symbol] |
| | in lab | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| Single- | The sludge | Operated | More than | 0.9 | 2.29 | [symbol] |
| chamber | from a | two-chamber | 1 year | | [symbol] /day | [symbol] |
| | local | MFC | | | | [symbol] |
| | waste water | | | | | [symbol] |
| | treatment | | | | | [symbol] |
| | plant | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| Single | Applying the | Operated | Over | 1.01 | [symbol] / | [symbol] |
| electrode | [symbol] | long-term | 6 months | | day | [symbol] |
| assembly | biofilm from | MEC | | | | [symbol] |
| ( [symbol] ) | a mature | | | | | [symbol] |
| | MEC | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| Single- | Scrapped | Operated | Over | 1.0 | 2.5 [symbol] / | [symbol] |
| chamber | from the | long-term | 6 months | | day | [symbol] |
| | anodes of | MEC | | | | [symbol] |
| | MECs | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| Single- | Anaerobic | Operated | Over 3 | 0.8 | 0.41 | [symbol] |
| chamber | sludge | long-term | months | | mmol/L/day | [symbol] |
| | digester | MEC under | | | | [symbol] |
| | located | 0.8 V | | | | [symbol] |
| | at the | | | | | [symbol] |
| | municipal | | | | | [symbol] |
| | waste water | | | | | [symbol] |
| | treatment | | | | | [symbol] |
| | plant | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |
| | | | | | | [symbol] |

[symbol]

indicates data missing or illegible when filed

Referring to Table 1, it can be seen that the start-up time of hydrogen production basically progresses for 1 month or up to 1 year when a low voltage is applied.

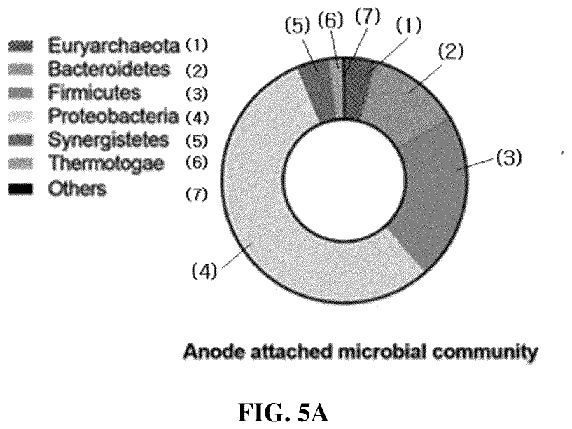

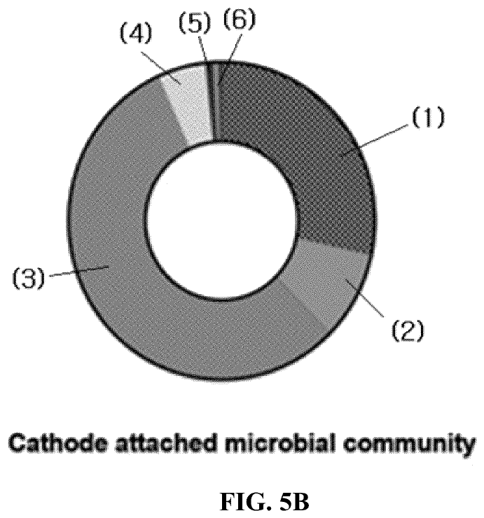

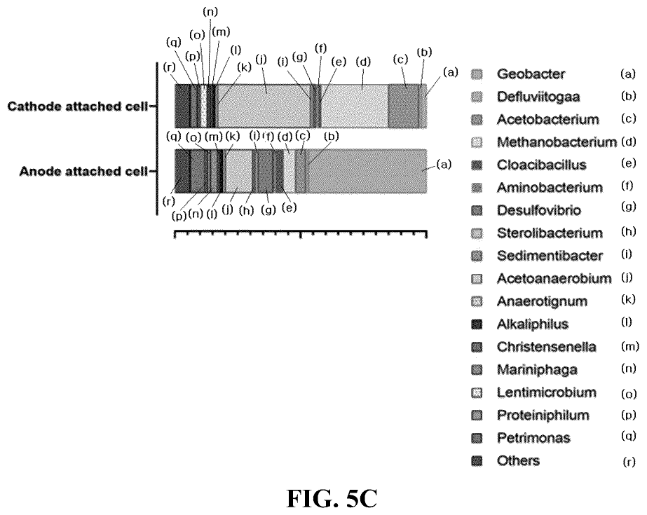

FIGS. 5A, 5B, and 5C show the results of (5A) microbial colonies attached to an anode electrode at a phylum level, (5B) microbial colonies attached to a cathode electrode, and (5C) microbial colonies attached to an anode electrode and cathode electrode at a genus level after operation of a single-chamber type microbial electrolysis cell system without an ion exchange membrane according to an embodiment of the disclosure (based on NGS analysis).

Referring to FIGS. 5A, 5B, and 5C, it can be confirmed that the microbial colonies present in the order of Proteobacteria, Firmicutes, and Bacteroidetes at the anode electrode, and in the order of Firmicutes, Euryarchaeota, and Bacteroidetes at the cathode electrode.

Proteobacteria attached to the anode electrode are known as bacteria that participate in a process of generating electricity by oxidizing representative organic matter, and include Geobacter. These species can contribute to transferring electrons by secreting oxidized substances on the electrode surface. Euryarchaeota attached to the cathode electrode decompose organic matter to produce methane, or are active in anaerobic environments and produce methane using carbon dioxide and hydrogen, and one of the major subgroups is known as Methanobacterium. Microbial colonies formed after methane inhibition can contribute to producing hydrogen.

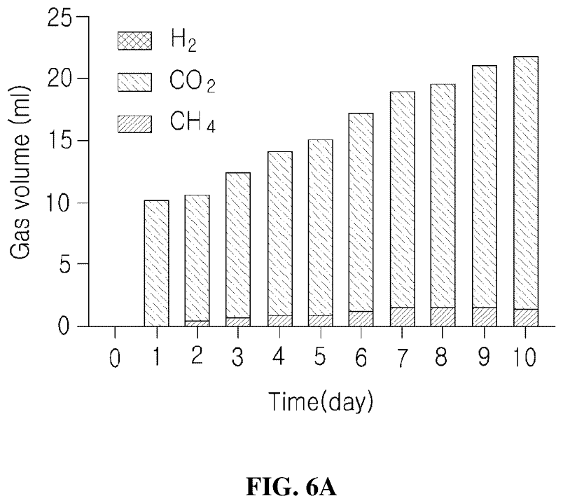

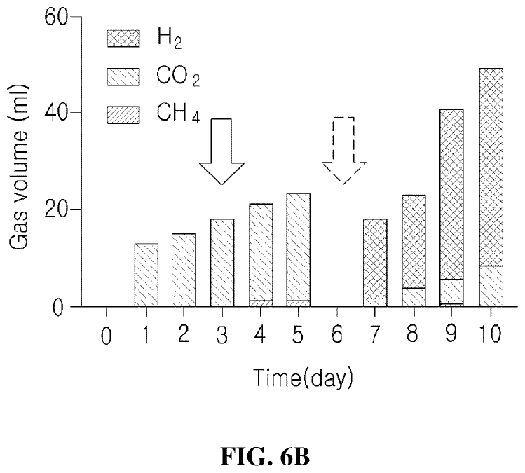

FIGS. 6A and 6B are results of hydrogen production in biogas production in a single-chamber type microbial electrolysis cell system without an ion exchange membrane according to an embodiment of the disclosure (6A) without application and (6N) after application of the technology proposed in the disclosure.

In the case of non-application (6A), the technology of the disclosure is not applied, and the reactor is operated by applying the applied voltage from the beginning of the reaction by applying the scheme reported so far.

In the case of application (6B), the reactor is operated under anaerobic conditions without applying potential at the beginning as suggested by the method of the disclosure, and then the potential is applied at the yellow arrow, and the red arrow indicates the point in time when the methane conversion bacteria are inhibited.

Referring to FIGS. 6A and 6B, when the disclosure is not applied, methane and carbon dioxide increase over time and hydrogen generation is almost non-existent, but when the disclosure is applied and the potential is applied and the methane conversion bacteria are inhibited, it can be confirmed that methane gas and carbon dioxide in the biogas decrease and hydrogen generation increases rapidly.

The description of the disclosure is for illustrative purposes, and those skilled in the art will understand that it can be easily modified into other specific forms without changing the technical idea or essential features of the disclosure. Therefore, the embodiments described above should be understood as being exemplary in all respects and not limiting. For example, each component described as a single type may be implemented in a distributed manner, and likewise, components described as distributed may be implemented in a combined form.

The scope of the disclosure is indicated by the following claims, and all changes or modifications derived from the meaning and scope of the claims and their equivalent concepts should be interpreted as being included in the scope of the disclosure.

### EXPLANATION OF REFERENCE NUMERALS

100: power supply unit
200: electrolysis chamber
201: anode electrode
202: cathode electrode
203: reference electrode
210: gas collection unit
220: organic matter supply unit
230: gas supply unit
240: sampling unit
250: solution replacement unit
300: water bath
400: stirring unit
500: gas-liquid separator
510: pump

What is claimed is:

1. A single-chamber microbial electrolysis cell system without an ion exchange membrane, the single-chamber microbial electrolysis cell system comprising:
a power supply unit;
a microbial electrolysis chamber connected to the power supply unit;
an electrode unit located within the microbial electrolysis chamber; and
a water bath connected to the microbial electrolysis chamber and configured to maintain a temperature,
wherein the microbial electrolysis chamber comprises:
a gas collection unit configured to confirm biogas production within the microbial electrolysis chamber;
an organic matter supply unit configured to supply an organic matter into the microbial electrolysis chamber;
a gas supply unit configured to supply an inert gas into the microbial electrolysis chamber;
a sampling unit configured to confirm consumption of the organic matter within the microbial electrolysis chamber;
a solution replacement unit configured to replace the organic matter within the microbial electrolysis chamber; and
a stirring unit configured to stir within the microbial electrolysis chamber, and
wherein the electrode unit comprises:
an anode electrode and a cathode electrode positioned to face each other within the microbial electrolysis chamber; and
a reference electrode located next to the anode electrode or the cathode electrode.

2. The single-chamber microbial electrolysis cell system according to claim 1, wherein the microbial electrolysis chamber includes a cylindrical or cassette-shaped chamber, and the cassette-shaped chamber is configured to expose only the cathode electrode to air to inhibit methanogens from forming on the cathode electrode, or allow the anode electrode and the cathode electrode to be separably attachable and detachable for electrode maintenance.

3. The single-chamber microbial electrolysis cell system according to claim 1, wherein in order to monitor biogas generated inside the microbial electrolysis chamber through the gas collection unit and inhibit methanogens from forming on the cathode electrode, exposing the cathode electrode to air, replacing an organic matter medium in a batch operation, removing a solution-phase methanogen by operating HRT within 24 hours in a continuous operation, or supplying a chemical inhibitor or antibiotic into the microbial electrolysis chamber is performed.

4. The single-chamber microbial electrolysis cell system according to claim 1, wherein a gas-liquid separator is further combined on one side of the microbial electrolysis chamber.

5. The single-chamber microbial electrolysis cell system according to claim 4, wherein the gas-liquid separator is configured to reduce a residence time of hydrogen produced in the microbial electrolysis chamber, thereby reducing an activity of microorganisms that consume the hydrogen.

6. A method for producing hydrogen and biogas using a single-chamber microbial electrolysis cell system without an ion exchange membrane, the method comprising:
supplying an organic matter to a chamber through an organic matter medium without applying an external voltage;
forming an electrochemically active microbial group by applying the external voltage to the chamber;
inhibiting methanogens from forming on a cathode electrode within the chamber; and
producing hydrogen and biogas.

7. The method according to claim 6, wherein in the supplying the organic matter to the chamber through the organic matter medium without applying the external voltage, anaerobic digestion conditions are achieved inside the chamber by supplying an inert gas into the chamber at a rate of 50 ml/min or less for 15 to 20 minutes, resulting in a production of the hydrogen and the biogas.

8. The method according to claim 6, wherein in the supplying the organic matter to the chamber through the organic matter medium without applying the external voltage, a temperature of the chamber is maintained in a range of 20 to 35° C.

9. The method according to claim 6, wherein the external voltage is applied in a range of 0.6 to 1.2 V using a two-electrode voltage application scheme.

10. The method according to claim 6, wherein the inhibiting the methanogens from forming on the cathode electrode within the chamber is achieved by exposing the cathode electrode to air, replacing the organic matter medium, removing a solution-phase methanogen by operating HRT within 24 hours in a continuous operation, exposing the cathode electrode to the air for 10 minutes to 1 hour, or supplying a chemical inhibitor or antibiotic.

11. The method according to claim 10, wherein the chemical inhibitor is selected from a first group including sodium 2-bromoethanesulfonate, 2-bromoethanesulfonate, Iodopropane, and lumazine, and the antibiotic is selected from a second group including neomycin sulfate, 2-chloroethane sulfonate, and 8-aza-hypoxanthine.

12. The method according to claim 6, wherein the producing the hydrogen and the biogas is performed by monitoring a potential of an anode electrode in the chamber based on a reference electrode in the chamber to check a concentration of the organic matter and replace the organic matter medium.

13. The method according to claim 6, wherein the producing the hydrogen and the biogas includes controlling carbon dioxide, methane and hydrogen contents of the biogas produced, depending on the external voltage of the single-chamber microbial electrolysis cell system without the ion exchange membrane, a temperature of the chamber, HRT (residence time of reactants in the chamber), or inhibition of methanogen and homoacetogen activity, to produce the biogas suitable for each intended use.

14. The method according to claim 7, wherein in the supplying the organic matter to the chamber through the organic matter medium without applying the external voltage, if the organic matter is a liquid with an organic acid and a gas containing carbon dioxide and methane, organic contaminants in the liquid containing the organic acid are removed within the chamber, and simultaneously, the carbon dioxide of the gas is combined with the hydrogen produced in the single-chamber microbial electrolysis cell system and converted into the methane, and
wherein highly soluble impurities including sulfur compounds, silicic acid, and odorous substances in the gas are removed while passing through the liquid within the chamber, increasing a content ratio of the hydrogen and the methane in the biogas produced in the single-chamber microbial electrolysis cell system.
